(19) United States
(12) Patent Application Publication
(10) Pub. No.: US 2007/0128702 A1
(43) Pub. Date: Jun. 7, 2007

(54) METHODS OF CREATING CONSUMABLE STRAINS AND COMPOSITIONS THEREOF

(75) Inventors: J. Mark Weber, Chicago, IL (US); Andrew Reeves, Chicago, IL (US); Igor A. Brikun, Forest Park, IL (US)

Correspondence Address: DYKEMA GOSSETT PLLC, 10 S. WACKER DR., STE. 2300, CHICAGO, IL, 60606, US

(73) Assignee: FERMALOGIC, INC., Chicago, IL (US)

(21) Appl. No.: 11/566,155

(22) Filed: Dec. 1, 2006

Related U.S. Application Data
(60) Provisional application No. 60/742,094, filed on Dec. 1, 2005.

(51) Int. Cl. C12P 19/62; C12N 15/74; C12N 1/21

(52) U.S. Cl. 435/76; 435/252.3; 435/252.35; 435/471

(57) ABSTRACT

Consumable biotech strain improvement products are presented, as well as methods of preparation and using them. The technology is based on reversible, single-crossover insertion vectors, such as plasmids or phage. Because the single crossover event is reversible in the absence of drug selection, the products cannot be maintained in a useful form without knowledge of the drug selection agent. Consumable strain improvement products can be constructed with 1st generation reverse engineering protections, having at least 25%-75% of the effectiveness of the equivalent traditional (permanent) strain improvement product under laboratory condition.

### CROSS-REFERENCE TO RELATED APPLICATIONS

This application claims priority to U.S. Ser. No. 60/742,094, filed Dec. 1, 2005, entitled, “METHODS OF CREATING CONSUMABLE BACTERIAL STRAINS AND COMPOSITIONS THEREOF,” and is incorporated by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable.

### TECHNICAL FIELD

The present invention relates to methods of making and using consumable bacterial strains, as well as compositions containing them.

### BACKGROUND

Historical Background

After a weekend vacation, Alexander Fleming returned to his laboratory to discover that one of his cultures of bacteria had been contaminated with mold. Not only was the plate contaminated, but the bacterial cells, Staphylococcus aureus, had lysed. Instead of throwing the contaminated plates away, Fleming observed that bacterial cell lysis occurred in an area next to the mold and hypothesized that the mold had made a product responsible for the death of the bacteria. He later was able to extract the diffusible substance from the mold, and penicillin was born.

Because antibiotics as a class of drugs are able to kill a broad spectrum of harmful bacterial pathogens, their use has revolutionized medicine, trivializing many diseases that had before taken millions of lives. For example, the plague, caused by infection with the Yersinias pestis bacterium, has laid claim to nearly 200 million lives and has brought about monumental changes, such as the end of the Dark Ages and the advancement of clinical research in medicine. Gentamycin and streptomycin are used to treat patients infected with plague, thus increasing the likelihood of survival. Erythromycins are used to treat respiratory tract and Chlamydia infections, diptheria, Legionnaires' disease, syphilis, anthrax and acne vulgaris. Erythromycins are also used to prevent Streptococcal infections in patients with a history of rheumatic heart disease.

Biological weapons are a real and current threat. Antibiotics are an important defense against the possible devastation such weapons can bring.

Antibiotics, among a vast array of other therapeutic and nutritional products, can be produced in enormous quantities thanks to genetic engineering of cells, especially of bacterial cells. Other important products that can be made by such fermentations include polypeptides, such as antigens for vaccines, antibodies and other therapeutic polypeptides; small molecules and small molecule inhibitors; and nutritional and industrial products, including isoflavones, sugars and alcohols.

For example, strain improvement technology for antibiotic producing strains began during the golden-age of antibiotic discovery in the 1940's. An empirical process of random mutation followed by large scale screening was used to find higher producing strains. Many of the antibiotic-producing strains in use today are descendants of the strains isolated during this early period.

The technology developed through the cooperation of industry and government during this period continued to be practiced relatively unchanged for many decades by private pharmaceutical companies in their strain improvement programs. Highly improved strains developed in-house were highly guarded from distribution to other companies.

Since the 1990's, strain improvement programs and other natural product related research at major pharmaceutical companies has been severely reduced or eliminated. However, the competition among big companies for the best strain for existing products, such as the antibiotic erythromycin, still continues. Today a larger percentage of the strain improvement work is out-sourced to specialty strain improvement.

The business of creating and selling biotech strain improvement technology for natural-product producing fermentations is in its early stages. Developers of this technology are historically pre-disposed to create strains with permanently improved trains, but if a strain can be created that has the same or nearly the same quality of strain improvement in a consumable format, it would be more beneficial to the biotech company to pursue this business approach.

The Problems with Out-Sourcing from the Biotech Company Perspective

The principal problems with out-sourcing, from the point of view of the small biotech (developer) company, are in control and distribution of the strain improvement technology. Distributing strains to countries that do not recognize US intellectual property rights also presents a problem. Pirating of strains is also a potential problem for both big and small companies.

The Problems with Out-Sourcing from the Pharmaceutical Company Perspective

Pharmaceutical companies do not want to pay a large up-front costs for technology that does not benefit their pharmaceutical process. Therefore they would like to test the new technology with low, up-front costs in case the technology does not work in their process. If the technology works, then payments to the developer company are justified.

### SUMMARY OF THE INVENTION

In a first aspect, the invention provides for methods of producing strains that have a target characteristic that the strain loses over time.

In a second aspect, the invention provides compositions containing strains that have target characteristic that the strain loses over time.

In a third aspect, the invention provides methods of doing business, wherein candidate strains that have target characteristics that the strain loses over time are provided to an end-user for evaluation.

In a fourth aspect, the invention provides methods of doing business wherein candidate strains that have target characteristics that the strain loses over time that have been evaluated by an end-user are repeatedly supplied to the end-user.

### DETAILED DESCRIPTION

The problem the developer of improved strains is leveraging fair value from strain improvement technology because of poor control of product distribution. The invention addresses the basic business problem with a technology-driven solution through the development of a strain improvement product in a “consumable” format.

Definitions

Biotech strain improvement technology refers to strain improvement introduced by recombinant DNA technology as opposed to being empirically introduced by random mutagenesis. The improvement can be transient or permanent.

Co-expressed, in the context of gene expression, means that two target traits are transcribed or translated or active simultaneously at some point during the life of the cell. The term is usually applied to two or more introduced genes. However, in some instances, co-expression will not occur unless the gene(s) is activated (e.g., a gene operably-linked to an inducible promoter); in this case, co-expression is applied in the context of when the inducible molecule(s) is present in the culture medium. Co-expressed genes can be genetically linked, but need not be.

Consumable refers to an item for sale intended to be used up and then replaced.

Consumable strain improvement product is defined by the characteristic of at least one transient genomic modification A reversible genetic modification introduced temporarily into the chromosome, for example, brought about by the introduction of a single-crossover event of an insertion plasmid. Also referred to as a plasmid-insertion construct; used in the consumable strain improvement format

End-user refers to a person or entity who uses a product. For the purposes of this application, an end-user may be the same as a customer, who might buy the product but does not necessarily use it, and thus has a more contractual meaning, wherein the term refers simply to a non-reseller.

Marker refers to one or more genes that are introduced exogenously into a cell. They are often carried by an insertion plasmid to indicate the presence of the plasmid in the genome. Markers are commonly genes that confer drug-resistance. A selectable marker is one that is to distinguish cells that have been successfully transformed, wherein exogenous DNA has been introduced, and confers upon the cells the ability to survive culture conditions in which their non-transformed counterparts die. Examples of selectable markers include antibiotic resistance marker genes, herbicide tolerant marker genes and metabolic marker genes. Screenable markers allow for the identification of successfully transformed cells, but do not confer a trait to the cells that allows for the survival of the cells from their non-transformed counterparts. Selection medium refers to a culture medium that contains a substance that allows for the identification of a cell carrying a selectable marker, while its non-transformed counterparts do not grow or die; screening medium allows for the identification of transformants, but does not prevent or kill non-transformed cells.

Prototype refers to a first or preliminary model of something.

Reverse engineering refers to the reproduction of another manufacturer's product following detail examination of its construction of composition.

Target trait means a characteristic that is brought about by a genetic modification, transient or permanent. Examples of target traits include those modifications that express or improve the expression of pharmaceutical or otherwise therapeutic substances (e.g., antibiotics, small molecules, small molecule inhibitors, antigens useful in vaccine formulations, therapeutic polypeptides (e.g., insulin, antibodies, portions of antibodies, engineered antibodies), etc.), or commercially important nutritional products (e.g., isoflavones, alcohols, sugars, etc.).

Traditional strain improvement product is defined by the characteristic of at least one permanent genomic modification A genetic modification introduced permanently into the chromosome, for example created by a double-crossover event following the introduction of an insertion plasmid. Also referred to as a gene-replacement construct. Two other types of permanent genomic modifications often used are transposon insertions or phage insertions (Kieser et al., 2000; Davison, 2002).

In the methods of the invention, strain improvement technology is usually introduced by recombinant DNA technology into a candidate organism. The strain has a temporal shelf-life. Once the improved strain is constructed, it is presented to the end-user for testing. Usually the end-user will test the strain on a small scale, and if successful, test the strain for compatibility in large-scale production formats. When microbial strains are being tested, testing often proceeds from shake flasks, followed by pilot plants, and finally the strain must show the improved production trait in the large scale commercial fermentors.

If after testing, the improved strain is not desired by the end-user, there will be no loss-control problems to consider since the improved strain loses its improvement effect and returns to its original pre-improvement condition during normal handling of the strain. An added benefit of the consumable format is that if the strain is inadvertently released into the environment and allowed to propagate, it reverts back to its natural, non-recombinant form.

If the end-user wishes to continue its commercial fermentations with the strain improvement effect, it simply purchases more modified strains from the developer company in the desired lot size. The developer company has an actual product to produce and sell on a periodic basis, rather than in the traditional method having a single product to sell in a one time deal. This keeps the two companies working together in a vendor-end-user relationship which fosters greater business interactions between the developer and the manufacturing companies.

The basis for “consumable” strain improvement technology relies on what has traditionally been seen as an unwanted feature of a widely used genetic technology called “insertion vectors” or “integration vectors”. Insertion vectors are plasmids or other circular DNA molecules such as phage, that have conditional replication combined with a selectable marker and at least one target DNA fragment for insertion of the plasmid into the chromosome. Such technology is applicable to almost all organisms, including prokaryotes (phages, viruses, bacteria) and eukaryotes (e.g, those numbering among the Opishtokonta, Amoebozoa, Plantae, Chromalveolate, Rhizaria and Excavata; Simpson and Roger, 2004). Examples include, but is certainly not limited to, Pseudomonas, Escherichia, Streptomyces, Saccharopolyspora, Bacillus, Ralstonia eutropha, Alcaligenes, Chromatium, Thiocystis, Saccharomyces, Yeasts, Clostridium, Thermobacillus, etc. Other examples of prokaryotes can be found in Balows, A., et al. (eds.). The Prokaryotes, 2nd ed. Springer-Verlag, New York. 1992, which is herein incorporated by reference.

The plasmid-based system, which relies on a Campbell-type homologous recombination, was originally developed and used in Bacillus subtilis in 1980 (Haldenwang et al.) and later adapted to S. erythraea in 1988 (Weber and Losick) and is still used today as a rapid method of generating genetic modifications in many different organisms, particularly bacteria.

Since their first use in 1980 for mapping the spoVG gene in B. subtilis, five additional new uses for integration vectors have been developed. Integration vectors are now also used for (1) gene knockouts, (2) gene amplifications, (3) chromosome walking, (4) gene fusions, and (5) ectopic (second-site) integration (Zeigler, 2002; Keiser et al., 2002).

Insertion vectors have proven useful for research purposes despite the unwanted feature of being inherently unstable in the bacterial chromosome. That is, insertion plasmids excise themselves spontaneously from the chromosome in a reversible reaction, leaving behind the original strain in its unaltered condition.

Molecular biologists have developed simple methods for handling strains carrying insertion plasmids to minimize the problems associated with their instability. One way to reduce the problems associated with instability to maintain selective drug pressure on the strain during an experiment, so that only individual cells that carry the insertion plasmid can survive. The cells that have lost the insertion plasmid are killed by the drug selective agent.

In a commercial environment, however, it is impractical (for cost and quality control issues) to maintain drug-selective pressure in large scale formats. While cells are generated during the course of the fermentation that have lost the plasmid conferring a desired trait, the half-life of plasmid loss is relatively long, the effect on yield is usually low. The end-user will not have knowledge of the drug-selection agent and so he is unable to propagate the improved strain for long until the newly propagated cells have all lost the plasmid and thus lost the desired trait. In order to regain the potency of the fully improved strain the end-user obtains them from the supplier.

Anti-Reverse Engineering

To protect the strain having a target characteristic, it must not being easily reverse engineered—an important aspect for the methods of the invention. The features of insertion plasmids that are most susceptible to reverse engineering are (1) the identification of the drug-resistance markers and (2) the identification of the target DNA sequences.

The use of novel and/or rarely used drug-resistance markers is the first line of defense. Examples of drugs that can be used as markers, or developed to be used as markers, is shown in Table 1. A preferred rarely used drug-resistance marker is viomycin resistance. Table 2 presents appropriate drugs and metabolites, as well as some providers and relative costs.

TABLE 1
Examples of resistance markers, their origins and GenBank accession numbers

| Gene | Function | Origin | Accessions |
|---|---|---|---|
| aac(3)IV | Apramycin acetyltransferase | Klebsiella pneumoniae | X99313 |
| aacCl | Gentamicin acetyltransferase | E. coli Tnl696 | U12338 X15852 |
| | | | U04610 |
| aacC7 | Paromomycin | S. rimosus | M22999 |
| | acetyltransferase | | |
| aacC8 | Neomycin acetyltransferase | S. fradiae | M55426 |
| aacC9 | Neomycin acetyltransferase | Micromonospora chalcea | M55427 |
| aadA | Spectinomycin/streptomycin | Pseudomonas plasmid R100.1 | M60473 |
| | adenyltransferase | | K02163 |
| ampC | P-lactamase | E. coli chromosome | V00277 |
| aph(4) | Hygromycin phosphotransferase | Klebsiella pneumoniae | V01499 |
| aphD | Streptomycin | S. griseus | X05647 |
| | phosphotransferase | | |
| aphE | Streptomycin | S. griseus | M37378 |
| | phosphotransferase | | |
| aphl | Aminoglycoside | S. fradiae | X02394 K00432 |
| | phosphotransferase | | |
| ardl | Aminoglycoside antibiotic A201A | S. capreolus | X84374 |
| | resistance | | |
| bar | Bialaphos resistance | S. hygroscopicus | X05822 |
| figal | P-galactosidase | S. lividans | M17359 |
| bla | P-lactamase | E. coli pBR322, pUC19 | JO1749 |
| | | | M77789 |
| ble | Bleomycin resistance (bleomycin | E. coli Tn5 | U00004 |
| | binding) | | |
| bleSh | Bleomycin binding | Streptoalloteichus hindustanus | |
| blmA | Bleomycin binding | S. verticillus | L26954/5 |
| blmB | Bleomycin acetyltransferase | S. verticillus | L26955 |
| cac | Capreomycin | S. capreolus | U13077 |
| | acetyltransferase | | |
| carA | Carbomycin efflux | S. thermotolerans | M80346 |
| carB | 23S rRNA methylase | S. thermotolerans | D31821 M1503 |
| cat | Chloramphenicol | E. coli Tn9 | V00622 X06403 |
| | acetyltransferase | pACYC184 | L08855 |
| | | pBR325 | |
| catSa | Chloramphenicol | S. acrimycini | |
| | acetyltransferase | | |
| cmlSl | Chloramphenicol efflux | S. lividans | X59968 |
| cmlv | Chloramphenicol export | S. venezuelae | U09991 |
| cph | Capreomycin | S. capreolus | U13078 |
| | phosphotransferase | | |
| cpt | Chloramphenicol | S. venezuelae | U09991 |
| | phosphotransferase | | |
| ere | Curromycin resistance | S. hygroscopicus | M28599 |
| drrA, B | Daunorubicin resistance | S. peucetius | M73758 U18082 |
| drrC | Daunorubicin resistance | S. peucetius | L76359 |
| EGFP | Green fluorescent protein | Aequorea victoria | U76561 |
| (gfp) | | | |
| ermE | 23S rRNA dimethylase | Saccharopolyspora erythraea | M37378 X51891 |
| ermSF | Old name for tlrA RNA N- | S. fradiae | M19269 |
| | methyltransferase | | |
| galK | Galactokinase | E. coli | D90714 |
| glkA | Glucose kinase | S. coelicolor | X65932 X98363 |
| grmMp | 16S rRNA methylase | Micromonospora purpurea | M55520 |
| grmMr | 16S rRNA methylase | Micromonospora rosea | M55521 |
| fiylR | Repressor of glycerol operon | S. coelicolor | X14188 |
| gyrB | Novobiocin resistant gyrase | S. sphaeroides | Z17304 |
| hur | Hydroxvurea resistance (tested in | S. aureofaciens | MSI 739 |
| | E. coli only) | | |
| ha | Hygromycin | S. hvgroscopicus | X99315 |
| | phosphotransferase | | |
| kciniA | 16S rRNA methylase | S. teiijimariensi.t | D13I7O |
| kinuB | 16S rRNA methylase | S. tenebrarius | M64625 |
| kamC | 16S rRNA methylase | Saccharopolyspora hirsute | M64626 |
| kan | 16S rRNA methylase | S. kananncelicits | M27488 |
| kan | 16S rRNA methylase | Micronumospora | — |
| | | echinospara | |
| kgmB | 16S rRNA methylase | S. tenebrarius | S6OIO8 |
| km | Aminoglycoside | E. coli Tn90i, | JO 1839 |
| | phosphotransferase | PACYC177. | V00359 |
| | | pUC4K | V00621 |
| ImrA | Lincomycin efflux | S. lincolneiisii | X59926 |
| imrB | 23S rRNA methylase | S. lincolncnsis | X62867 |
| Inn | 23S rRNA methylase | S. lividaiis | M74717 |
| luxA, B | Luciferase | Vibrio harvevi | X58791 |
| imlh | Malate dehydrogenase | Thermits flavus | X54073 |
| nurA.B | Mitomycin C resistance | S. htvendulue | L29247 |
| met | Other tyrosinases | S. ventziiflot | M2O422 |
| | | S. lincolnensis x | X957O3 |
| | | galbus | X95705 |
| melCl.Cl | Tyrosinase | S. imtibioticus | MII582 |
| melCl.CI | Tyrosinase | S. glaucescens | MI 1302 |
| mer | Mercury resistance | S. liridnns | X65467 |
| mmr | Methylenomycin efflux | S. coelicolor | Ml 8263 |
| nniA.B | Mithramycin resistance. nitrA. ATP- | S. argillaleus | U43537 |
| | binding. mtrB membrane protein | | |
| myrB | 23S rRNA methylase | Micromonospora griseorubida | E07944 |
| | | | D14532 |
| natl | Nourseothricin acetyltransferase | S. noursei | S60706 |
| | | | X73149 |
| neo | Aminoglycoside phosphotransferase | E. coli TnJ | U00004 |
| nmr | Neomycin resistance | S. cyanogenus pSB24.2 | X03756 |
| | | | M32513 |
| nonR | Macrotetrolide nonactin, tetranactin | S. griseus | M75853 |
| | efflux | | |
| nsh | 23S rRNA methylase, nosiheptide | S. actuosus | U75434 |
| | resistance | | |
| oleA, B | Oleandomycin resistance | S. antibioticus | L36601 |
| oleC | Oleandomycin efflux | S. antibioticus | L06249 |
| oriT | Origin of transfer | Plasmid RP4 = RK2 | L27758 (nt |
| | | | 50590-51384) |
| otrA | Oxytetracycline sequestration | S. rimosus | X53401 |
| | (similar to EF Tu) | | 132939 |
| otrB | Oxytetracycline efflux | S. rimosus | AF061335 |
| pac | Puromycin acetyltransferase | S. alboniger | X76855 |
| pat | Phosphinothricin acetyltransferase | S. viridochromogenes | A02804 |
| ptr | Multidrug resistance | S. pristinaespiralis | X84072 |
| pur8 | Puromycin resistance | S. alboniger | X76855 |
| rphSl | Ribostamycin phosphotransferase | S. ribosidificus | M22126 |
| rpsL | Ribosomal protein (Strs) | S. roseosporus | U60191 |
| spcN | Spectinomycin phosphotransferase | S. flavopersicus | U70376 |
| sph | Streptomycin phosphotransferase | S. glaucescens | X78976 |
| srmB | Spiramycin efflux | S. ambofaciens | X63451 |
| ter(fd) | Transcriptional terminator | E. coli phage fd | V00602 |
| tet | Tetracycline efflux | E. coli pBR322 | JO1749 |
| tetSl | Tetracycline efflux | S. lividuns | M74049 |
| tipA | Thiostrepton-inducible protein | S. fivkkins | Y08949 |
| tlrA | N-methyltransferase | S. fradiae | Ml 9269 |
| lirB | Methyltransferase | S. fradiae | AF055922 |
| | | | AJ00997I |
| tlrC | Tylosin efflux resistance | S. fradiae | M57437 |
| tlrD | Tylosin constitutive 235 rRNA | S. fradiae | X9772I |
| | monomethylase | | |
| nn-B2J | Tetronasin efflux | S. longisporuflavm | X73633 |
| iruR | Transfer gene regulator | pSN22, S. niirifaciens | D1428I |
| tsr | 235 rRNA methylase | S. azureus | X54219 |
| | | | X02392 |
| tsr | Thiostrepton resistance | S. laurentii | L39I57 |
| vph | Viomycin phosphotransferase | S. viuaceus | X02393 |
| | | | X99314 |
| xylE | Catechol-2.3-dioxygenase | Pseudomonas | U03992 |
| | | | JO1845 |

TABLE 2
Antibiotics, antimetabolites, and suppliers

| Name (Synonyms) | Class, properties | Suppliers' and relative price▪ |
|---|---|---|
| Amikacin | Aminoglycoside | Sigma▪▪ |
| Apramycin | Aminoglycoside | Duchefa, Sigma▪ |
| Bialaphos | Glutamine synthetase inhibitor, | Meiji Seika, Duchefa▪▪▪ |
| | herbicide | |
| Blasticidin S | Used as fungicide against rice | Invitrogen, CAYLA▪▪▪▪▪ |
| | blast | |
| Bleomycin | Cross-resistance with | Boehringer, Calbiochem. Duchefa, |
| | phleomycin and zeocin | Sigma▪▪▪▪▪▪▪ |
| Butirosin | Aminoglycoside | Sigma, Duchefa▪▪▪ |
| Capreomycin | Peptide, cross-resistance with | Sigma▪▪ |
| | viomycin | |
| Carbomycin | Macrolide | Pfizer |
| (Magnamycin ®) | | |
| Chloramphenicol | Antibacterial | Sigma, Duchefa, etc.▪ |
| Ciprofloxacin | Gyrase inhibitor | |
| Clindamycin | Semi-synthetic lincosamide | Sigma▪▪▪▪▪ |
| Daunomycin/daunorubicin | Anthracycline, anti-cancer | Sigma▪▪▪▪▪▪ |
| | agent | |
| Destomycin | Aminoglycoside used as feed | |
| | additive | |
| Erythromycin | Macrolide | Sigma, Duchefa▪ |
| Fortimicin | Aminoglycoside | |
| Fosfomycin (phosphomycin) | Antibacterial | Sigma▪ |
| Fusidic acid | Antibacterial | Sigma▪ |
| Geneticin (G418) | Aminoglycoside | Boehringer, Clontech, Gibco/BRL, |
| | | Invitrogen, Sigma. A.G. Scientific▪▪▪ |
| Gentamicin | Aminoglycoside | Boehringer, Calbiochem, Duchefa, |
| | | Sigma▪▪ |
| Hydroxyurea | Antineoplastic agent | Sigma▪ |
| Hygromycin B | Best selection on low-salt | A.G. Scientific Boehringer, |
| | media, light sensitive | Calbiochem, Clontech, CAYLA, |
| | | Duchefa, Invitrogen, Sigma▪▪▪▪ |
| Kanamycin | Aminoglycoside, best selected | Boehringer, Calbiochem, Clontech, |
| | on low-salt media | Duchefa, Sigma▪ |
| Kasugamycin | Aminoglycoside | Sigma▪ |
| Lincomycin | Lincosamide, used in | Sigma, Duchefa▪▪ |
| | preference to erythromycin to | |
| | select ermE in S. coelicolor | |
| Lividomycin | Aminoglycoside | |
| Methylenomycin | Cyclopentane | |
| Minamycin | Macrolide | |
| Mitomcin C | Anticancer agent | Boehringer, Calbiochem, |
| Nalidixic acid | Gyrase inhibitor; | Duchefa, Sigma▪ |
| | streptomycetes are naturally | |
| | resistant, E. coli is sensitive | |
| Neomycin | Aminoglycoside | Calbiochem, Sigma▪ |
| Nonactin | Macrotetrolide (no medical use) | Sigma▪▪▪▪▪ |
| Nosiheptide | Peptide, similar to thiostrepton | |
| Nourseothricin | Similar to streptothricin | |
| Novobiocin | Gyrase inhibitor | Boehringer, Serva▪ |
| Oleandomycin | Macrolide | Serva Pfizer▪ |
| (Oleandocyn ®) | | |
| Pactamycin | No medical use | |
| Paromomycin | Aminoglycoside | Sigma, Duchefa▪ |
| Penicillins | Most streptomycetes are | Many |
| | naturally resistant to penicillins | |
| Phleomycin | Similar to bleomycin | Sigma, CAYLA▪▪▪▪▪▪ |
| Phosphinothricin | Glutamine synthetase inhibitor | Duchefa▪▪▪▪▪ |
| Puromycin | Nucleoside | Calbiochem, Clontech, Sigma, CAYLA, |
| | | A.G. Scientific▪▪▪▪▪ |
| Racemomycin | Streptothricin type; active | |
| | against mycobacteria | |
| Ribostamycin | Aminoglycoside | Sigma▪▪▪ |
| Rifampicin | Light-sensitive | Boehringer, Duchefa, Sigma▪▪▪ |
| Siomycin | Peptide, similar to thiostrepton | |
| Sisomicin | Aminoglycoside | Sigma▪▪ |
| Spectinomycin | Antibacterial | Boehringer, Duchefa, Sigma▪ |
| Spiramycin | Macrolide | Sigma▪ |
| Streptogramin B (Synercid ®) | Antibacterial | Rhone-Poulenc Rorer |
| Streptomycin | Antibacterial | Calbiochem, Duchefa▪ |
| Streptothricin | Similar to nourseothricin | |
| Tetracycline | Light-sensitive | Calbiochem, Boehringer, Duchefa, Sigma▪ |
| Tetranactin | Macrotetrolide (see nonactin) | |
| Tetronasin | Polyether (feed additive) | |
| Thiopeptin | Peptide, similar to thiostrepton | |
| Thiostrepton | Peptide; poor selection on MS | Calbiochem▪▪ |
| | (=SFM) | Sigma |
| Tobramycin | Aminoglycoside | Duchefa, Sigma▪▪▪ |
| Tuberactinomycin | Similar to viomycin | |
| Tylosin | Macrolide | Sigma▪ |
| Viomycin | Peptide; cross-resistance with | Sigma▪▪▪▪▪▪ |
| (Viocin, Florimycin) | capreomycin I A, B; most | Research Diagnostics |
| | active on low-salt media | |
| Zeocin | Similar to bleomycin | Invitrogen, CAYLA▪▪ |

Suppliers of antibiotics: the blocks (▪) indicate the relative prices of the antibiotics; some of the prices are very high only because the quantities sold are small and the demand for the substances is low. Samples for research may be obtainable from the antibiotic producers directly.

The second line of defense is to remove those elements that facilitate recovering the polynucleotide fragment that contains the target characteristic(s). For example, in bacteria, if the E. coli origin of replication if left in the strain facilitates rapid recovery of the plasmid from the partially digested chromosome and subsequent transformation of the ligated DNA with ampicillin selection into E. coli where the plasmid could be easily characterized. Alternatively, the E. coli origin of replication could be left in the construct as long as the traditional resistance gene was removed and replaced with an alternate drug resistance marker such as viomycin resistance or an entirely new marker.

Other mechanisms to foster protection from reverse engineering can be used. For example, if the strain improvement effect is exerted through the concerted transcription of two to five genes, their arrangement in the genome can be altered. For example, if the genes are usually clustered, they could be separated and re-inserted into the chromosome at ectopic (neutral second) sites, and each expressed separately and held in place through selective pressure, each with a different antibiotic. If any one of the insertions is lost, the desired characteristic is also lost.

Other types of modifications can be used; falling under the six uses of insertion plasmids described above. For example, insertion plasmids can create gene knockouts, and gene amplifications. Just about any modification can be created through the use of an insertion plasmid.

Determining the Stability of Strains Carrying Insertion Plasmids

The stability of the insertion plasmid in the chromosome may affect the quality of the strain improvement effect. Experiments are performed to determine the stability of varying size insertion plasmids and then determine how much of an effect stability plays on the target characteristic. The rate of spontaneous excision (i.e., the instability) of insertion plasmids from the chromosome depends upon the size of the target DNA fragment in the insertion plasmid, the organism, and the insertion site (Metzenberg et al., 1991).

Method
1. Construct insertion plasmids carrying the polynucleotides which encode the target characteristic.
2. Transform plasmids into wild-type host organism, using selection, to create strains.
3. Analyze each strain for presence of inserts.
4. Culture strains without selection.
5. Sample cultures over time and perform a time course analysis to determine the proportion of cells carrying the insertion plasmid.
6. The proportion of drug-resistant colonies per unit volume are plotted vs. time to determine the half-life of the insertion plasmid in the chromosome.

The methods for analyzing for drug resistance and the target characteristic are known to those of skill in the art and vary according to organism and the effect of the target characteristic (Ausubel et al., 1987).

Comparing Improved Strains in the Consumable vs. the Permanent Format

Construction of a permanently modified strain is done using known techniques (Ausubel et al., 1987).

Method
1. Culture the permanently modified strain and the temporal strains.
2. Assay for the desired characteristic(s) for each strain and compare.

In the methods of the invention, consumable product strains are about 25%-100% efficient as their permanent counterparts (that is, one improved using traditional methods). More preferably, the consumable product strains are at least 50% efficient, and more preferably, at least 75% efficient as the permanent counterpart.

Protecting Against Reverse Engineering of the Consumable Strain Improvement Technology

The consumable strain improvement product is designed to give the owner of the strain improvement technology control over its sale and distribution. The key to maintaining the strain without its losing its strain improvement effect is by propagating the strain in the presence of the proper drug-selection agent. Other key information that could be gained by reverse engineering regards the identity of the cloned target DNA sequences and the origins of replication on the plasmid.

As an example, the modification of a bacterial strain is provided. In this strategy, the commonly used resistance genes are substituted for the less commonly used viomycin resistance gene. The ColE1 origin of replication (ori) is replaced by conditional R6Kγ E. coli ori. Plasmids containing this origin of replication do not replicate in the commonly used E. coli host strains, but require a special E. coli host strain, R6Kγ-5.

Other more stringent protections include eliminating the E. coli origins of replication completely and create plasmid constructions exclusively in the host organism.

Kits

The consumable products can be included in a kit, container, pack, or dispenser together with instructions for growth and, if appropriate, induction of the target characteristic. When supplied as a kit, the different components can be packaged in separate containers. Such packaging can permit long-term storage without losing the activity of the components. For example, such kits can contain containers of spores and powdered media. In other embodiments, the kits include the consumable product with instructions for use; in other embodiments the kits include an order form to re-order the product, or directions to obtaining additional product, either from a website, via electronic mail, telephone, facsimile, or any other appropriate channel of communication.

Kits may also include reagents in separate containers that facilitate the execution of a specific test, such as tests for the target characteristic.

Containers or vessels The reagents included in the kits can be supplied in containers of any sort such that the life of the different components are preserved and are not adsorbed or altered by the materials of the container. For example, sealed glass ampoules may contain lyophilized buffer that has been packaged under a neutral non-reacting gas, such as nitrogen. Ampoules may consist of any suitable material, such as glass, organic polymers, such as polycarbonate, polystyrene, etc., ceramic, metal or any other material typically employed to hold reagents. Other examples of suitable containers include bottles that may be fabricated from similar substances as ampoules, and envelopes, that may consist of foil-lined interiors, such as aluminum or an alloy. Examples of containers include test tubes, vials, flasks, bottles and syringes. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, etc.

Instructional materials Kits can also be supplied with instructional materials. Instructions may be printed on paper or other substrate, and/or may be supplied as an electronic-readable medium, such as a floppy disc, CD-ROM, DVD-ROM, Zip disc, videotape, audiotape, mini-disc, cassette tape or provided by calling a prescribed telephone number. Detailed instructions may not be physically associated with the kit; instead, a user may be directed to an Internet web site specified by the manufacturer or distributor of the kit, or supplied as electronic mail.

Business Methods

In one embodiment, a potential end-user requests the consumable product for testing for her purposes. The supplier supplies the consumable product, either as live cultures, frozen cultures, or in an inactive state (e.g., spores, seeds, etc.). The end-user then tries the product in her application until either the strain no longer expresses the desired characteristic, the application is determined to be inappropriate for the strain, or the strain is applicable to the application and more strain is requested. In the case of the latter, the end-user then orders the quantities she needs for her application, replacing the strain as its target characteristic wanes. If the strain is inappropriate, the end-user is free to use the strain (subject to any agreements, such as contracts or licenses, etc.), until the desired characteristic dissipates, or, more usual, the strain is appropriately disposed of.

### EXAMPLES

The following examples exemplify a specific embodiment wherein S. erythraea is modified according to the methods of the invention to increase production of erythromycin.

### Example 1 Construction of Insertion Plasmid pFL2212 and Improved Strain FL2385

Background Plasmid pFL2212 contains a 6.8 kb fragment of the S. erythraea chromosome (Fermalogic, Inc.; Chicago, Ill.). It is the insertion plasmid for the consumable strain improvement product, strain FL2385 (Fermalogic). Plasmid pFL2212 contains the mutAB region of the S. erythraea chromosome. This plasmid contains the 6.8 kb methylmalonyl-CoA mutase region from S. erythraea. The plasmid also contains the Streptomyces origin of replication, pIJ101, which has lost the ability to replicate autonomously in S. erythraea. The tsr gene encodes the thiostrepton-resistance gene is also on the plasmid and is used in the selection of transformants after protoplast transformation. neo encodes a promoter-less neomycin resistance gene, also included on the plasmid. ColE1 is the E. coli replication origin; Apr, represents the ampicillin resistance gene

Method A cosmid library of S. erythraea chromosomal DNA was prepared in a SuperCos cosmid vector (Stratagene, La Jolla, Calif.). Approximately 600 recombinant cosmids were screened by PCR for the presence of the mutAB region using primers designed based on the DNA sequence information deposited by Luz-Madrigal et al., 2002 (Genbank accession no. AY117133 (SEQ ID NO:1)). The primer sequences were: gntRF1-5′-GTCGAATTCGCCGTCACCGTCGACCCCAA-3′ (SEQ ID NO:2) and gntR1-5′-GTCGGATCC CAGCATCAGCGCTCCCGGA-3′ (SEQ ID NO:3). Two cosmids, 5G10 and 6E7, were found containing the mutAB operon. Cosmid 6E7 was used for DNA sequencing of the mutAB flanking regions using a primer-walking method. Cosmid 6E7 was also used to sub-clone a fragment of DNA containing the five genes shown in FIG. 4between the EcoRI and BamHI sites. This fragment was cloned into pFL8 to create plasmid pFL2212. S. erythraea protoplasts were transformed with pFL2212 with selection for thiostrepton resistance. Single thiostrepton-resistant colonies were isolated tested in shake flask fermentation. These strains were designated FL2385.

### Example 2 Fermentation Results with the Improved Strain FL2385

Method Fermentations were performed in un-baffled 250-ml Erlenmeyer flasks with milk-filter closures. The flasks were incubated at 32.5° C. and 65% humidity on an Infors Multitron Shaker having 1-inch circular displacement. Seed cultures containing a carbohydrate-based medium (and no thiostrepton) were prepared on the same shaker under the same growth conditions. Seed cultures were inoculated from fresh spores prepared on fresh sporulation agar plates containing thiostrepton at 10 micrograms/ml. Fermentations were inoculated with 1.25-ml of a 42 h seed culture into 25-ml of oil-based media. Thiostrepton was not added to any seed or fermentation media. Fermentations were grown for 5 days; their volumes were then corrected for evaporation through the addition of water before being further analyzed.

Results Strain FL2385 produced erythromycin in excess of 35% above that which can be achieved with the wild-type (white) S. erythraea strain when grown under optimal fermentation conditions including using an oil-based medium.

Conclusions The strain improvement trait of FL2385 was significant and occurred in the absence of drug selection in the fermentation or seed media. The only point at which FL2385 was exposed to drug selection was in the preparation of the spores used to inoculate the fermentation seed medium. The insertion plasmid pFL2212 therefore was sufficiently stable in the chromosome to produce a significant strain improvement effect.

### Example 3 (Prophetic)

A new strain, FL2385P, will be created that is genetically equivalent to the current prototype consumable strain FL2385. This strain will have a duplication of the 1st generation gene cluster region, but will be a permanent genomic modification created by a gene-replacement technique rather than a single-crossover technique.

Next a bank of plasmids will be created with different sized target DNA sequences that will allow for the measurement of the half-life of the strain improvement effect in strains made with the transient (consumable) technology.

### Example 4 (Prophetic) Determine the Stability of Strains Carrying Insertion Plasmids

Rationale The stability of the insertion plasmid in the chromosome can affect the quality of the strain improvement effect. Experiments can be performed to determine the stability of varying size insertion plasmids and then determine how much of an effect stability plays on erythromycin production during the course of the fermentation. The rate of spontaneous excision (i.e. the instability) of insertion plasmids from the chromosome depends upon the size of the target DNA fragment in the insertion plasmid, the organism, and the insertion site (Metzenberg et al., 1991)

Method

1. Construct insertion plasmids of different sizes, pFL2212 A-G.

2. Transform plasmids pFL2212 A-G into wild type S. erythraea (strain FL11635), using thiostrepton selection, to create strains FL2385 A-G, respectively.

3. Produce thiostrepton-resistant spores of Strains FL2385 and FL2385 A-G

4. Analyze the spores of each strain to show that the spores are 100% thiostrepton resistant, and therefore all carrying the insertion plasmid.

5. Perform shake flask fermentations on each of the seven strains. See Preliminary Results for a description of the fermentation method. Seed cultures and fermentation cultures will not contain thiostrepton since in the commercial setting it would be impractical to add these drugs to large-scale fermentors.

6. Seed cultures and fermentations will be sampled daily and a time course analysis will be performed to determine the proportion of cells carrying the insertion plasmid. The stability analysis will be performed by plating the culture samples on sporulation agar and incubating the plates at 32° C. for 10 days until the plates is fully sporulated. Spores will be harvested and diluted in 20% aqueous glycerol to obtain single colonies on sporulation agar plates. A sample of one hundred colonies will be screened for the presence of the insertion plasmid by patching colonies on agar containing thiostrepton at 10 micrograms/ml, and also on plates containing no thiostrepton. Alternatively, equivalent volumes of diluted spore suspensions will be plated on thiostrepton and no-thiostrepton agar to calculate plasmid loss from the difference in colony numbers on the two plates. Additionally, it may prove useful to place a visual marker gene on the insertion plasmid to aid in the counting of colonies.

7. The proportion of thiostrepton-resistant colonies per unit volume will be plotted vs. time to determine the half-life of the insertion plasmid in the chromosome.

### Example 5 (Prophetic) Compare Improved Strains in the Consumable vs. the Permanent Format

To know whether the consumable strain improvement product with the transient genomic modification can achieve the level of strain improvement that is attainable by a permanently modified strain, equivalent strains will be constructed in both formats and then compared them directly in fermentations.

Construction of the permanently modified strain will be done using the gene replacement technique using standard techniques. An earlier study (Reeves et al., 2002) described the eryCI-flanking region; this is a suitable site for insertion of the second copy of the mutAB gene pair by a gene replacement.

Method
1. Perform PCR to amplify two contiguous 2.6 kb ery cluster flanking regions (accession no. AF487998) using the pMW3 template (containing the eryCI-flanking region; Reeves et al., 2002) and primers with restriction sites engineered at their 5′ ends. One set of primers will have HindIII sites at their 5′ ends and the other will have EcoRI sites.
2. Clone first the PCR product with HindIII ends into the unique HindIII site on pFL2212.
3. Confirm the correct orientation of the cloned fragment by sequence analysis. This construct will be designated pFL2238.
4. Clone the second 2.6 kb PCR product containing the EcoRI ends into the unique EcoRI site on pFL2238.
5. Confirm the correct orientation of the cloned fragment by sequence analysis. This construct will be designated pFL2239.
6. Use PCR to amplify the kanamycin resistance gene contained on pUC4K (Pharmacia Biotech, Piscataway, N.J.) engineered with XbaI sites at the 5′ ends.
7. Clone the kanamycin resistance gene into the unique XbaI site on pFL2239. Select for kanamycin resistant E. coli clones. This construct will be designated pFL2240.
8. Transform protoplasts of S. erythraea wild type strain FL11635 with pFL2240 and select with kanamycin to generate double crossover (gene replacement) strains in a one-step process (Reeves et al., 2002).
9. Distinguish between double and single crosses by patching transformants onto E20A agar plates containing thiostrepton, kanamycin and no antibiotic. Subject kanamycin-resistant, thiostrepton-sensitive strains to further analysis.
10. Determine site of double crossover (gene replacement) using PCR and the chromosomal DNA from kanamycin-resistant, thiostrepton-sensitive strains. The site of homologous recombination could be two regions: the ery cluster flanking region or the native methylmalonyl-CoA mutase region. Use PCR primers that amplify a unique junction fragment between the kanamycin resistance gene and the ery cluster flanking sequences.
11. Perform shake flask fermentations comparing the two strains, the consumable strain improvement product with the transient genomic modification (FL2385) and the strain containing a permanent insertion of the same genes at a second (ectopic) site (FL2385P). Spores of FL2285 will be prepared on sporulation agar containing thiostrepton (10 micrograms/ml) and spores of strain FL2385P will be prepared on sporulation agar without thiostrepton.
12. Perform scale-up fermentations with strains FL2385 and FL2385P in 3 L stirred-jar fermentors using the same oil-based fermentation medium.

### Example 6 (Prophetic) Protection Strategy Against Reverse Engineering of the Consumable Strain Improvement Technology

The consumable strain improvement product is designed to give the owner of the strain improvement technology control over its sale and distribution. The key to maintaining the strain without its losing its strain improvement effect is by propagating the strain in the presence of the proper drug-selection agent. Other key information that could be gained by reverse engineering regards the identity of the cloned target DNA sequences and the origins of replication on the plasmid. Our initial protections for our first generation strain improvement technology described here will involve substituting the commonly used thiostrepton-resistance gene and kanamycin resistance gene for the less commonly used viomycin-1-resistance gene. The ColE1 origin of replication (ori) will also be replaced by conditional R6Kγ-4 E. coli ori. Plasmids containing this origin of replication do not replicate in the commonly used E. coli host strains, but require a special E. coli host strain, R6Kγ-5. Also the ampicillin-resistance gene will be eliminated because the viomycin resistance gene is a useful selectable marker for both Streptomyces and E. coli.

Method
1. Isolate plasmid pProprietary-3 from S. lividans FL20. pProprietary-3 contains the viomycin resistance gene, the thiostrepton-resistance gene (tsr), and the pIJ101 Streptomyces origin of replication. It lacks the ampicillin resistance gene (bla), a conditional E. coli origin, and the S. erythraea methylmalonyl-CoA mutase (mmCoA) region.
2. PCR the E. coli origin of replication, R6Kγ-4 (Reeves et al., 2004), using high fidelity taq polymerase and primers that have engineered PstI sites at their 5′ ends.
3. Clone the R6Kγ-4 origin of replication into the unique PstI site of pProprietary-3. This construct will be designated pFL2241.
4. Electroporate pFL2241 into R6Kγ-5 E. coli strain. Select for viomycin resistance. This confirms two features of the construct: function of the R6Kγ-4 origin of replication and viomycin resistance.
5. Clone into the unique PvuII site on pFL2241 a 6.7 kb BamHI+EcoRI blunt ended fragment from pFL2212 containing mutA, mutB, meaB, and gntR. This construct will have a functional viomycin resistance gene, an inactivated thiostrepton-resistance gene, a functional R6Kγ-4 origin of replication and the S. erythraea methylmalonyl-CoA mutase region. This construct will be designated pFL2242.
6. Protoplast transform pFL2242 into the S. erythraea wild type strain with selection for viomycin resistance.
7. Perform shake flask fermentations with strain FL2385C and compare erythromycin production with strain FL2385P, the strain containing a permanent second site (ectopic) copy of the methylmalonyl-CoA mutase region.
8. Measure the proportion of thiostrepton-resistant colonies per unit volume plotted vs. time to determine the half-life of the insertion plasmid in the chromosome.

The examples here presented are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

### REFERENCES

Ausubel, F. M., R. Brent, R. E. Kingston, D. D. Moore, J. G. Seidman, J. A. Smith, and K. Struhl. 1987. Current protocols in molecular biology. John Wiley & Sons, New York.
Balows, A., H. G. Truper, M. Dworkin, W. Harder, and K.-H. Schleifer (eds.). The Prokaryotes, 2nd ed. Springer-Verlag, New York. 1992
Bibb M J, White J, Ward J M, Janssen G R. The mRNA for the 23S rRNA methylase encoded by the ermE gene of Saccharopolyspora erythraea is translated in the absence of a conventional ribosome-binding site. Mol Microbiol. 1994 November; 14(3):533-45.
Davison J. Genetic tools for Pseudomonads, Rhizobia, and other Gram-Negative Bacteria (2002) BioTechniques 32:386-401
Haldenwang W G, Banner C D, Ollington J F, Losick R, Hoch J A, O'Connor M B, Sonenshein A L. Mapping a cloned gene under sporulation control by insertion of a drug resistance marker into the Bacillus subtilis chromosome. J Bacteriol. 1980 April; 142(1):90-8.
Hopwood, D. A., Bibb, M. J., Chater, K. F. & 7 other authors (1985). Genetic Manipulation of Streptomyces: a Laboratory Manual. Norwich: John Innes Foundation.
Kieser T, M. J. Bibb, M. J. Buttner, K. F. Chater & D. A. Hopwood (2000) PRACTICAL STREPTOMYCES GENETICS. Norwich: The John Innes Foundation
Metzenberg A B, Wurzer G, Huisman T H, Smithies O. Homology requirements for unequal crossing over in humans. Genetics. 1991 128:143-61
Reeves A R, Weber G, Cernota W H, Weber J M. (2002) Analysis of an 8.1-kb DNA fragment contiguous with the erythromycin gene cluster of Saccharopolyspora erythraea in the eryCI-flanking region. Antimicrob Agents Chemother 46:3892-3899
Simpson, A G, and Roger, A J. (2004) The real ‘kingdoms’ of eukaryotes. Curr Biol. 14(17):R693-6.
Weber J M, Losick R. The use of a chromosome integration vector to map erythromycin resistance and production genes in Sacchropolyspora erythraea (Streptomyces erythraeus). Gene. 1988 Sep. 7; 68(2):173-80.
Zeigler D R (2002) Bacillus Genetic Stock Center Catalog of Strains, Seventh Edition Vol 4: Integration vectors for Gram-positive organisms. The Ohio State University, Columbus, Ohio 43210.

1. A method of providing a consumable biological strain having at least one target trait to an end-user, comprising:
modifying a genome of at least one cell to express the at least one target trait, wherein the at least one target trait is co-expressed with a selectable marker;
identifying the modified cell in a selection medium comprising a substance to which the marker confers resistance; and
providing the modified cell to the end-user;
wherein the cell is provided without identifying the marker to the end-user, and wherein culturing the cell in the absence of the substance results in the target trait being lost over time.

2. The method of claim 1, wherein modifying the genome comprises introducing a single-crossover of a plasmid, wherein the plasmid carries the target trait.

3. The method of claim 1, wherein the modified cell is provided as part of a kit.

4. The method of claim 1, wherein the modified cell is a eukaryote or prokaryote.

5. The method of claim 4, wherein the modified cell is a prokaryote and is one selected from the group consisting of Streptomyces, Saccharopolyspora, Bacillus, Pseudomonas, Escherichia, Ralstonia, Alcaligenes, Chromatium, Thiocystis, Clostridium, and Thermobacillus,

6. The method of claim 1, wherein the marker confers resistance to amikacin, apramycin, bialaphos, blasticidin s, bleomycin, butirosin, capreomycin, carbomycin, chloramphenicol, ciprofloxacin, clindamycin, daunomycin, daunorubicin, destomycin, erythromycin, fortimicin, fosfomycin, fusidic acid, geneticin, gentamicin, hydroxyurea, hygromycin b, kanamycin, kasugamycin, lincomycin, lividomycin, methylenomycin, minamycin, mitomycin c, nalidixic acid, neomycin, nonactin, nosiheptide, nourseothricin, novobiocin, oleandomycin, pactamycin, paromomycin, penicillins, phleomycin, phosphinothricin, puromycin, racemomycin, ribostamycin, rifampicin, siomycin, sisomicin, spectinomycin, spiramycin, streptogramin b, streptomycin, streptothricin, tetracycline, tetranactin, tetronasin, thiopeptin, thiostrepton, tobramycin, tuberactinomycin, tylosin, viomycin, viocin, florimycin and zeocin.

7. The method of claim 1, wherein the marker is one selected from the group consisting of aac(3)IV, aacCl, aacC7, aacC8, aacC9, aadA, ampC, aph(4), aphD, aphE, aphl, ardl, bar, figal, bla, ble bleSh, blmA, blmB, cac, carA, carB, cat, catSa, cmlSl, cmlv, cph, cpt, ere, drrA,B, drrC, EGFP, (gfp), ermE, ermSF, galK, glkA, grmMp, grmMr, fiylR, grB, hur, ha, kciniA, kinuB, kamC, kan, kan, k gmB, km, ImrA, imrB, Inn, luxA,B, imlh, nurA.B, met, melCl.Cl, melCl.CI, mer, mmr, nniA.B, myrB, natl, neo, nmr, nonR, nsh, oleA,B, oleC, oriT, otrA, otrB, pac, pat, ptr, pur8, rphSl, rpsL, spcN, sph, srmB, ter(fd), tet, tetSl, tipA, tlrA, lirB, tlrC, tlrD, nn-B2J, iruR, tsr, tsr, vph, and xylE.

8. The method of claim 1, wherein the target trait results in the cell producing a therapeutic or nutritional substance.

9. The method of claim 8, wherein the therapeutic substance is a small molecule, a small molecule inhibitor, an antigen, an antibody or portion thereof, an antibiotic, or a polypeptide.

10. The method of claim 8, wherein the nutritional substance is a vitamin, a sugar, an alcohol, an isoflavone, or a polypeptide.

11. The method of claim 10, wherein the nutritional substance comprises an isoflavone.

12. The method of claim 1, wherein the cell is S. erythraea, the target trait is increased erythromycin production, and the marker is thiostrepton resistance.

13. A method of doing business, wherein a consumable biological strain having at least one target trait is supplied to an end-user on an on-going basis, the method comprising:
modifying a genome of at least one cell to express the at least one target trait, wherein the at least one target trait is co-expressed with a selectable marker;
identifying the modified cell in a selection medium comprising a substance to which the marker confers resistance;
providing the modified cell to the end-user;
wherein the cell is provided without identifying the marker, and wherein culturing the cell in the absence of the substance results in the target trait being lost over time; and wherein additional modified cells are provided to an end-user upon request.

14. The method of claim 13, wherein modifying the genome comprises introducing a single-crossover of a plasmid, wherein the plasmid carries the target trait.

15. The method of claim 13, wherein the modified cell is provided as part of a kit.

16. The method of claim 13, wherein the modified cell is a eukaryote or prokaryote.

17. The method of claim 17, wherein the modified cell is a prokaryote and is one selected from the group consisting of Streptomyces, Saccharopolyspora, Bacillus, Pseudomonas, Escherichia, Ralstonia, Alcaligenes, Chromatium, Thiocystis, Clostridium, and Thermobacillus,

18. The method of claim 13, wherein the marker confers resistance to amikacin, apramycin, bialaphos, blasticidin s, bleomycin, butirosin, capreomycin, carbomycin, chloramphenicol, ciprofloxacin, clindamycin, daunomycin, daunorubicin, destomycin, erythromycin, fortimicin, fosfomycin, fusidic acid, geneticin, gentamicin, hydroxyurea, hygromycin b, kanamycin, kasugamycin, lincomycin, lividomycin, methylenomycin, minamycin, mitomycin c, nalidixic acid, neomycin, nonactin, nosiheptide, nourseothricin, novobiocin, oleandomycin, pactamycin, paromomycin, penicillins, phleomycin, phosphinothricin, puromycin, racemomycin, ribostamycin, rifampicin, siomycin, sisomicin, spectinomycin, spiramycin, streptogramin b, streptomycin, streptothricin, tetracycline, tetranactin, tetronasin, thiopeptin, thiostrepton, tobramycin, tuberactinomycin, tylosin, viomycin, viocin, florimycin and zeocin.

19. The method of claim 13, wherein the marker is one selected from the group consisting of aac(3)IV, aacCl, aacC7, aacC8, aacC9, aadA, ampC, aph(4), aphD, aphE, aphl, ardl, bar, figal, bla, ble, bleSh, blmA, blmB, cac, carA, carB, cat, catSa, cmlSl, cmlv, cph, cpt, ere, drrA,B, drrC, EGFP, (gfp), ermE, ermSF, galK, glkA, grmMp, grmMr, fiylR, grB, hur, ha, kciniA, kinuB, kamC, kan, kan, k gmB, km, ImrA, imrB, Inn, luxA,B, imlh, nurA.B, met, melCl.Cl, melCl.CI, mer, mmr, nniA.B, myrB, natl, neo, nmr, nonR, nsh, oleA,B, oleC, oriT, otrA, otrB, pac, pat, ptr, pur8, rphSl, rpsL, spcN, sph, srmB, ter(fd), tet, tetSl, tipA, tlrA, lirB, tlrC, tlrD, nn-B2J, iruR, tsr, tsr, vph, and xylE.

20. The method of claim 13, wherein the target trait results in the cell producing a therapeutic or nutritional substance.

21. The method of claim 20, wherein the therapeutic substance is a small molecule, a small molecule inhibitor, an antigen, an antibody or portion thereof, an antibiotic, or a polypeptide.

22. The method of claim 20, wherein the nutritional substance is a vitamin, a sugar, an alcohol, an isoflavone, or a polypeptide.

23. The method of claim 22, wherein the nutritional substance comprises an isoflavone.

24. The method of claim 13, wherein the cell is S. erythraea, the target trait is increased erythromycin production, and the marker is thiostrepton resistance.


SEQUENCE LISTING (3 sequences)

SEQ ID NO: 1 - DNA, 5894 bp, Saccharopolyspora erythraea
ggttctcggagtcggcggtcccggtgcggtgcaggcggctgcgccaaggcgcaccggctgccgggcgcgggaccgacgagctgacactggtgggtggtcgttcggtgcacctcgcggtgcgggacgtcccgcgcggcgtgctcgggatcgcctgggactgggactgaggcgcccggcggacgctctgccctgtccggctgcgacaagcgtcacacgatccccgggccgggccgcaccggcctaccatcctgttcatggtggcgcactcgacgacgagcgacgggccggagctgcccctggcggccgagttccccgagcccgcccggcagcagtggcggcaacaggtggagaaggtcctgcgcaggtcgggtctgctgcccgagggcaggcccgcgccggagccggtcgaggacgtgctcgccagcgccacctacgacggcatcaccgtgcacccgctctacaccgagggtcccgcatccagcggcgtcccgggcctggcgccctacgtgcgcggcagccgggcgcagggctgcgtcagcgagggctgggacgtccgccagcaccacgcccaccccgacgcctcggagaccaaccgcgagatcctggccgacctctacaacggcacgacctcgctgtggctggagctcgggccgaccgggctgccggtggactcgctggccgacgccctcgaaggcgtccacctggacatgatcggcgtcgtgctcgacgccggtgacgaggcggcgcgggccgcgtcggcgttgctggagctcgcgcgggagcagggggtgcggcccagcgcgctgcgcgccaacctgggcgccgacccgctgagcacctgggctcgcaccgggcaggaacgcgacctgggcctcgccgccgaggtcgccgcgcactgcgcgtcgcacccgggcctgcgcgcgatcaccgtcgacggcctgccctaccacgaggcgggcggctccgacgccgaggagctcggctgctcgatcgccgcgggcgtcacctacctgcgggtgctggccggtgagctcggtgccgaggccgcgagcgggctgctggagttccgctacgccgccaccgccgaccagttcctgaccatcgccaagctgcgcgcggcccgcaggctgtgggagcgggtgacgcgggagatcggcgtcgccgagcgcgcgcagctccagcacgcggtcacctcctcggcgatgctgacgcgccgcgacccgtgggtgaacatgctgcgcaccacgatcgccacgttcgccgcaggcgtgggcggcgcgcggtcggtcaccgtgcgcccgttcgacgccgcgatcgggctgccggaccccttctcccggcgcatcgcccgcaacacccagtcgctgctgctggaggagtcgcacctggcgcaggtgatcgacccggcgggcggttcctggtacgtcgagacgctgaccgacgaactggcgcacaaggcgtgggagtggttccggcgcatcgaggccgagggcgggctgcccgccgcgctgcgctcgggtctggtggccgaccggctcgccgagacctggcagcggcgccgggacgccgtcgcccaccgcaccgacccgatcaccggcgtcaccgagttcccgaacctcgaagaacccgcgctgcgacgcgaccccgcgcccgagccgctgtcgggcggcctgccccgccaccgctacgccgaggacttcgagcggctgcgcgacgcctccgacgcccacctcgccgaaaccggtgcgcgcccgaaggtcttcctcgccacgctcggttcgctcgccgagcacaacgcccgcgcgtcgttcgcccgcaacctcttcggcgcgggcgggctggaaaccccggacgccgggcccacggagtccacagaggacgtggtgaaggcgttcgccggctcgggcacgccggtggcctgcctgtgctcgggtgaccggatctacggtgagcacgcggaggaaaccgcccgcgcgctccgggaggcgggggccgaccaggtgctgctggccggctcgctcgaggtgcccggcgtcgacggccgggtgttcggcgggtgcaacgccctcgaagtcttgcaggacgtccaccgcaggttgggagtgcagcagtgaccgcccacgagcacgaaccgatccccagcttcgccggcgtggagctgggcgagcccgcccccgcgcctgccgggcggtggaacgacgcgctgctggccgagaccggcaaggaggccgacgccctggtgtgggaggcgcccgagggcatcggcgtcaagccgctctacaccgaggccgacacccgcgggctggacttcctgcgcacctacccgggaatcgcgccgttcctgcgcggcccgtacccgacgatgtatgtcaaccagccgtggacggtgcgccagtacgcggggttctccaccgccgagcagtccaacgccttctaccgccgcaacctcgccgccgggcagaagggcctgtcggtggccttcgacctggccacccaccgcggctacgactccgaccacccgcgcgtcggcggtgacgtcggcatggcgggcgtggcgatcgactccatctatgacatgcgccggctcttcgacggcatcccgctggacaggatgagcgtgtcgatgacgatgaacggcgccgtgctgccggtgatggcgctctacatcgtcgccgccgaggaacagggcgtggcgccggagaagctggccgggaccatccagaacgacatcctcaaggagttcatggtccgcaacacctacatctacccgccgcagccgtcgatgcggatcatctccgacatcttcgcctacgcctcgcggcggatgccgaagttcaactcgatctccatctccggctaccacatccaggaggccggggcgaccgccgacctggagctggcctacaccctcgcggacggcgtggagtacctgcgcgccgggcggcaggcgggcctggacatcgactccttcgccccgcggctgtcgttcttctggggcatcgggatgaacttcgcgatggaggtcgccaagctgcgcgcggcccggctgctgtgggccaagctggtcaagcgcttcgagccgtcggacccgaagtcgctgtcgctgcgcacccactcgcagacctcgggctggtcgctgaccgcccaggacgtctacaacaacgtcgtgcgcacgtgcgtggaggcgatggccgccacccagggccacacccagtcgctgcacaccaacgccctggacgaggcgctggcgctgccgaccgacttctccgcgcgcatcgcccgcaacacccagctggtgctccagcaggagtccggcaccacccgcgtcatcgacccgtggggcggctcgcactacatcgagcggctgacccaggacctcgccgaacgcgcgtgggcccacatcaccgaggtcgaggacgccggcggcatggcccaggccatcgacgccggtatcccgaagatgcgcatcgaggaggccgccgcgcggacgcaggcgcgcatcgactccggccgccagccgctcatcggcgtcaacaagtaccgctacgacggcgacgagcagatcgaggtcctcaaggtcgacaacgccggcgtgcgggcccagcagctggacaagctgcggcggctgcgcgaggaacgcgactccgaggcgtgcgagaccgcactgcgcaggctgaccggcgccgccgaggccgcgctggaggacaaccggcccgacgacctcgcgcacaacctgctgacgctggccgtggacgccgcgcggcacaaggccaccgtcggcgagatctccgacgcgctggagaaggtcttcggccgccactccggccagatccgtacgatttccggcgtgtaccgggaggagtcgggtacctcggagtcgctggagcgcgcccgccgcaaggtcgaggagttcgacgaggcagagggcaggcgcccgcgcatcctggtggccaagatgggccaggacggccacgaccgcggccagaaggtcatcgccaccgccttcgccgacatcggcttcgacgtcgacgtgggcccgctgttccagaccccggccgaggtcgcccgccaggcggtcgagtccgacgtgcacgtcgtcggggtgtcgtcgctggccgcgggccacctgacgctggtgcccgcgctgcgcgacgagctggccgggctcggccgctccgacatcatgatcgttgtcggcggcgtgatcccgcccgccgacttcgacgcgctgcgccagggcggagccagcgcgatcttcccgccgggaaccgtgatcgccgacgccgcgctcggactgctcgaccagctccgcgcggtgctcgaccaccccgcgcccggcgagcctgccggcgagtcggacggcgcccgaggcggttcccccggcgagacgtcgagcgcgggctgaccatgccgcgcgagatcgacgtccaggactacgccaagggcgtgctcggcggctcgcgcgccaagctggcgcaggcgatcacgctggtggagtcgaccagggccgagcaccgcgcgaaagcccaggaactgctcgtcgagctgctgccgcacagcggtggggcgcaccgggtgggcatcaccggcgtgcccggcgtcggcaagtcgacgttcatcgagtcgctgggcacgatgctgaccgcgcaggggcaccgggtcgcggtgctggcggtcgacccgtcgtccacgcgcagcggcggcagcatcttgggcgacaagacgcggatgcccaagttcgcctccgactccggcgcgttcgtgcggccctccccctcggcgggcacgctcggcggcgtcgcgcgcgcgacccgcgagacgatcgtgctgatggaggcggccggattcgacgtcgtgctcgtggaaacggtgggcgtcggccagtccgaggtcgccgtggcgggaatggtcgactgcttcctgctgctgacgctggcccgcaccggcgaccagttgcagggcatcaagaagggtgtgttggagctggccgaccttgtcgcggtgaacaaggccgacggaccgcacgagggcgaggcgcgcaaggcggcccgcgagctgcgcggcgcgctgcggctgctgaccccggtcagcacgtcgtggagacccccggtggtgacctgcagcggcctgaccggagcgggcctggacacgctctgggagcaggtcgagcagcaccgcgccaccctcaccgagaccggcgagctggccgagaagcgcagccgccagcaggtcgactggacctgggcgctggtgcgcgaccagctcatgtccgacctgacccggcacccggcggtgcgccgcatcgtcgacgaggtcgaatccgacgtgcgggccggggaactgaccgcgggcatcgccgccgagcggctgctcgacgccttccgggagcgctgatgctggccgtcaccgtcgaccccaactccgctgtcgcaccgttcgagcaggtgcgcacgcagatcgcgcagcagatcaacgaccgcgtcctgccggtcggaaccaagctgcccaccgtgcgccggctggcggccgacctcggcatcgcggccaacaccgcggccaaggcctaccgcgagctggagcaggcgggactgatcgaaacccgtggccgcgcgggaaccttcgtgggctcggcgggcgagcgcagcaacgagcgcgcggccgaggccgccgccgagtacgcccggaccgtcgccgcgctgggcatcccccgcgaggaggcacttgccatcgtgcgcgcggccctgcgcgcgtagggccgccctgcgggcgtagcgcggccctgcgggcgtagcgcggccctgcgggcttggcgcggcccgggcgggttcagcgcttcgcgcggcgccgcgcgagacggcgcggggccacctgctcggcctgctccccctggatcc

SEQ ID NO: 2 - DNA, 29 bp, Artificial sequence
  Primer
gtcgaattcgccgtcaccgtcgaccccaa

SEQ ID NO: 3 - DNA, 28 bp, Artificial sequence
  Primer
gtcggatcccagcatcagcgctcccgga